(19) United States
(12) Patent Application Publication
(10) Pub. No.: US 2003/0147848 A1
(43) Pub. Date: Aug. 7, 2003

(54) Use of n-desulfated heparin for treating or preventing inflammations

(76) Inventor: Jian-Guo Geng, Shanghai (CN)

Correspondence Address: BIRCH STEWART KOLASCH & BIRCH, PO BOX 747, FALLS CHURCH, VA, 22040-0747, US

(21) Appl. No.: 10/297,768

(22) PCT Filed: Jun. 8, 2001
(86) PCT No.: PCT/CN01/00922

(30) Foreign Application Priority Data
Jun. 9, 2000 (CN) 0016404

(51) Int. Cl. A61K 38/24; A61K 38/20; A61K 38/19; A61K 31/727

(52) U.S. Cl. 424/85.1; 424/85.2; 514/12; 514/56

(57) ABSTRACT

The invention relates to the use of N-desulfated heparin for treating or preventing inflammation, which is based on the experimental results in animal acute abdominal inflammation model and animal bleed model. The N-desulfated heparin's anti-inflammation activity is better than or equal to that of the low molecular weight heparin, and has low activity of anti-coagulant. From a series of N-desulfated heparin of different N-sulfur-containing, a sample of the best anti-inflammation activity and the lowest anti-coagulant activity was selected. The invention solved the problem of bleeding in the use of heparin for treating of inflammation, and provided a new pathway to use heparin to prevent and treat inflammation.

This application is the national phase under 35 U.S.C. §371 of PCT International Application No. PCT/CN01/00922 which has an International filing date of June 8, 2001, which designated the United States of America.

### FIELD OF THE INVENTION

The present invention relates to the pharmaceutical use, especially about using N-desulfated heparins, which have significantly reduced anticoagulant activities and preserve anti-inflammation activity, for prevention and treatment of inflammation. The invention further relates to methods and means for testing heparin and heparin derivatives with significantly reduced anticoagulant activities for therapeutic purposes.

### DESCRIPTION OF BACKGROUND AND RELATED ART

Heparin is a highly sulfated natural polysaccharide that is first described by McLean in 1916 and has been used clinically as an anticoagulant for more than 50 years [McLean, Circulation 19, 75-78 (1959)]. It is synthesized in the endoplasmic reticulum as proteoglycans attached to the protein serglycine [Toledo and Dietrich, Biochim. Biophys. Acta 498, 114-122 (1977)]. Heparin is an intracellular product found in the secretary granules of mast cells often in complexation with basic proteins. In response to certain external signal, it is released exclusively from mast cells of the lung, intestine and liver during their degranulation. The released soluble heparin carries negative charges and is know to bind to more than one hundred proteins including many growth factors and chemokines. Heparin can be isolated, in a relative inexpensive way, from highly vasculaturized tissues, such as porcine or bovine intestinal mucosa, or less commonly, from bovine lung tissue [Conrad, Heparin-Binding Proteins, Academic Press, San Diego (1998)].

Heparin is a mixture of polydisperse, structurally similar, unbranched polymers made up of repeating units of alternating disaccharides containing hexuronate (either β-D-glucuronic acid or α-L-iduronic acid) and α-D-glucosamine (either N-sulfated or N-acetylated). They are joined by (1,4) glycosidic linkages. The major sites where sulfation may be present are at the 2-O position of the iduronic acid residues as well as the 2-N position and 6-O position of the glucosamine residues. In addition, it is occasionally sulfated on either the 2-O position of glucuronic acid residues or the 3-O position of the disulfated glucosamine residues [Tyrrell et al., Adv. Pharmacol. 46, 151-208 (1999)]. The linear glycosaminoglycan chains are covalently bound to serine residues of core proteins. The heparin polymer may contain 20-100 monosaccharide units per polysaccharide chain, and due to the long repeats of (ser-gly)ₙ in the protein sequence, a high density of heparin chains can be attached to the serglycine core [Ruoslahti, J. Biol. Chem. 264, 13369-13372 (1989)]. However, the number and position of GAG chains vary with the protein core.

Besides heparin, there has another sulfated polysaccharide, heparan sulfate, which is structurally similar to heparin and contains all of the structural motifs found in heparin. In contrast to heparin, it is normally secreted from cells. It is distributed ubiquitously on the surface of most animal cells and as a major component of the basement membranes in extracellular matrix. Heparin and heparan sulfate are members of a family of polysaccharides termed “Glycosaminoglycans,” or GAGs [Hook et al., Annu. Rev. Biochem. 53, 847-869 (1984)]. In addition to heparin and heparan sulfate, this family includes chondroitin-sulfate, dermatan sulfate and hyaluronic acid.

Heparin and heparan sulfate have the most heterogeneous structures in the GAG family. Heparan sulfate typically has a low level of N- and O-sulfation and retains more of the original N-acetylglucosamine and glucuronate residues. Compared to heparin, heparan sulfate generally has fewer and shorter GAG chains which can be attached to a variety of core proteins, often in conjunction to chondroitin sulfate chains [Kjellen and Lindahl, Ann. Rev. Biochem. 60, 443-475 (1991)]. It can also be attached directly to the cell surface (via either a transmembrane domain of the core protein or a phosphatidyl-inositol likage) or they may be bound by specific receptors at the cell surface [Gallagher, Curr. Opin. Cell Biol. 1, 1201-1218 (1989)]. It has reported that different cells can have distinct subtypes of heparan sulfate chain, suggesting a possibility that they may play roles in mediating cellular interactions [Kim et al., Mol. Biol. Cell 5, 797-805 (1994); Engelmann et al., Biochim. Biophys. Acta Mol. Cell Res. 1267, 6-14 (1995); Archer et al., J. Anat. 189, 23-35 (1996)].

Compared to heparan sulfate, heparin is more highly sulfated and contains a greater percentage of iduronate residues which have been epimerized from glucuronate. Due to the differences in composition and the extent of sulfation, heparin is more highly charged than heparan sulfate. The higher percentage of iduronate residues in heparin also increases the relative flexibility of the polymer. This flexibility coupled with an increase in the presence of additional electrostatic interactions is presumed to provide a greater biological activity associated with iduronate-containing GAGs as compared to glucuronate containing counterparts [Casu et al., Trends Biochem. Sci. 13, 221-225 (1988)].

The most widely accepted and commercially exploited functions of heparin are as an anticoagulant. This action of heparin resides in its ability to regulate the activity of an endogenous coagulation cofactor, antithrombin-III (AT-III), which inhibits many serine proteases involved in the coagulation cascade [Bourin and Lindahl, Biochem. J. 289, 313-330 (1993)]. Heparin can interact with AT-III, via a specific high-affinity pentasaccharide sequence that represents only a minor portion of any heparin chain, to form a complex that inhibits thrombin and Factor Xa much more effectively than AT-III alone. Heparin can also interact with another serine protease inhibitor, heparin cofactor II (HC-II), to further potentate the inhibition of thrombin.

Beyond its well-recognized anticoagulant activity, heparin and heparan sulfate have various non-anticoagulant actions. Among them are anti-inflammatory roles which include prevention of leukocyte adhesion [Tangelder and Arfors, Blood 77, 1565-1571 (1991); Ley et al., Am. J. Physiol. 260, H1667-H1673 (1991); Arfors and Ley, J. Lab. Clin. Med., 121, 201-202 (1993); Teixeira and Hellewell, Br. J. Pharmacol. 110, 1496-1500 (1993); Nelson et al., Blood. 82, 3253-3258 (1993); Seeds et al., J. Lipid Mediators 7, 269-278 (1995); Kitamura et al., Eur. Surg. Res. 28, 428-435 (1996); Weber et al., J. Cereb. Blood Flow Metab. 17, 1221-1229 (1997); Giuffre et al., J. Cell Biol. 136, 945-956 (1993)] and activation [Pasini et al., Thromb. Res. 35, 527-537 (1984); Bazzoni et al., J. Lab. Clin. Med. 121, 268-275 (1993); Riesenberg et al., Br. Heart J. 73, 14-19 (1995); Ahmed et al., Am. J. Respir. Crit. Care Med. 155, 1848-1855 (1997)], inhibition of complement activation [Weiler et al., J. Immunol. 148, 3210-3215 (1992); Teixeira et al., J. Leukocyte Biol. 59, 389-396 (1996)], maintenance of endothelial wall competence and integrity and protection of vascular endothelial cells from a number of damaging substances, such as chemokines, histamine, bradykinin, bacterial endotoxin, lysosomal cationic proteins and oxygen free radicals [Engelberg, Semin. Thromb. Hemost. 11, 48-55 (1985); Hiebert and Liu, Semin. Thromb. Hemost. 17(suppl 1), 42-46 (1991); Tanaka et al., Nature 361, 79-82 (1993); Webb et al., Proc. Natl. Acad. Sci. USA 90, 7158-7160 (1993)].

For example, heparin and heparan sulfate are known to bind to cell adhesion molecules P-selectin, L-selectin and Mac-1 (CD11b/CD18) and to inhibit leukocyte adhesion mediated by these molecules [Skinner et al., Biochem. Biophys. Res. Commun. 164, 1373-1379 (1989); Skinner et al., J. Biol. Chem. 266, 5371-5374 (1991); Nelson et al., Blood. 82, 3253-3258 (1993); Norgard-Sumnicht et al., Science. 261, 480-483 (1993); Diamond et al., J. Cell Biol. 130, 1473-1482 (1995); Giuffre et al., J. Cell Biol. 136, 945-956 (1997); Koenig et al., J. Clin. Invest. 101, 877-889 (1998)]. Heparan sulfate, synthesized by endothelial cells [Castillo et al., Biochem. J. 247, 687-693 (1987); Kinsella and Wight, Biochemistry 27, 2136-2144 (1988)], can bind to various chemokines and therefore optimally localize these chemotactic cytokines [Tanaka et al., Nature 254, 79-82 (1993); Furie and Randolph, Am. J. Pathol. 146, 1287-1301 (1995)]. Heparin and heparan sulfate can bind to many biochemical constituents on endothelial cells and thus restore the negative charge of the endothelial cell surface made positive following tissue injury [Engelberg, Semin. Thromb. Hemost. 11, 48-55 (1985); Hiebert and Liu, Semin. Thromb. Hemost. 17(suppl 1), 42-46 (1991)].

In literature, heparin can by modified chemically in many ways. These modified ones have been proved to be invaluable for the structural and functional studies. They include periodate oxidation, N-desulfation, N-deacetylation, modifications of N-unsubstituted heparinoids, 2-O-desulfation, 6-O-desulfation, carboxyl reduction and derivatization, epimerization, sulfate migration, and oversulfation [Conrad, Heparin-Binding Proteins, Academic Press, San Diego (1998)].

Tiozzo and his colleagues have reported the effect of desulfation of heparin on its anticoagulant. N-desulfated heparin derivatives, compared with starting heparin, have significantly reduced anticoagulant activity, but the anticoagulant activity of heparin and its derivatives has no absolute linear relationship with the N-sulfate content of heparin chains. [Tiozzo et al., Thromb. Res. 70: 99-106 (1993)].

Using chemical modifications, it has been shown that the non-anticoagulant heparins could inhibit rat arterial smooth muscle cell proliferation in vivo [Guyton et al., Circ. Res. 46, 625-634 (1980)]. The 2-O-desulfated and 3-O-desulfated heparins had reduced anticoagulant activities, but preserved their heparanase-inhibitory, angiostatic, anti-tumor and anti-metastatic properties. (Masayuki et al., U.S. Pat. No. 5,795, 875 (1997); Lapierre et al., Glycobiology 6, 355-366 (1996)].

In addition, Ahmed and his colleagues have reported that they could generate non-anticoagulant heparin by collection of the low affinity fraction of the intact, non-chemical modified heparin from affinity resins conjugated with antithrombin III. The non-anticoagulant heparin thus prepared could inhibit antigen-induced acute bronchconstriction, airway hyperresponsiveness, and mass cell degranulation [Ahmed et al., Am. J. Respir. Crit. Care Med. 155:848-1855 (1997)].

Heparin and low molecular heparin can be used to treat inflammatory diseases, but have danger side effect of hemorrhage in the tissue due to the potent anticoagulant activity, which limits their extensively clinical use on inflammatory patients. Many chemical modification methods was patented to reduced their anticoagulant activity while preserve other biological properties. An example of O-desulfated heparin derivatives is described in U.S. Pat. No. 5,795,875. It shows this O-desulfated heparin derivatives, are useful for treating various diseases including inflammation, consists of substantially unfragmented 6-O desulfated heparin, or 6-O desulfated heparin fragments, but still retains 5-30% anticoagulant activity of the starting heparin. Another example of O-desulfated heparin, preferably 2-O, 3-O desulfated heparin is described by Holme et al. U.S. Pat. No. 5,296,471, retains 5.5-23% anticoagulant activity of the starting heparin. But these O-desulfated heparins still have potent anticoagulant activity.

It has been known that N-desulfated heparin has significant reduced anticoagulant activity, but there are few reports of the effect of removing N-sulfate of heparin chain on its anti-inflammation activity, so it is deserved to research the anti-inflammation activity of N-desulfated heparin, particularly in prevention and treatment of the inflammation diseases.

### PURPOSE OF THIS INVENTION

Therefore, the purpose of this invention is to provide a pharmaceutical use of N-desulfated heparin in prevention and treatment of inflammation, the anti-inflammation activity of N-desulfted heparin is better than or equal to LMH, and has a significant reduced anticoagulant activity. The present invention provides a good possibility to use N-desulfated heparin for prevention and treatment of inflammation better.

Furthermore, present invention, prevention and treatment of inflammation by N-desulfated heaprin, is based on the chemical modification method to remove the N-sulfate of heparin chain and get eight heparin derivatives, then screening the best anti-inflammation and lowest anticoagulant activity sample through the acute peritonitis mouse model and the bleeding time assay. The results indicate the effect of desulfation on its anti-inflammation and anti-coagulant activity, N-desulfated heparins have better anti-inflammation activity than LMH, while have even lower anti-anticoagulant activity.

Starting heparin was chemically modified to remove the N-sulfate, referred to the publications [Nagasawa et al., Carbohydr. Res. 36,265-271,1976; Inoue and Nagasawa, Carbohydr. Res. 46,87-95(1976)], getting N-desulfated heparin with different mount of N-sulfate. The anticoagulant activity of heparin, LMH and all N-desulfated derivatives was determined. Table 1 shows the results of absolute, relative content of N-sulfate and the aPTT time.

TABLE 1

| Heparin and its | Absolute N-sulfate | Relative N-sulfate | 2aPTT |
|---|---|---|---|
| derivatives | content (%) | content (%) | (μg/ml) |
| UFH | 3.25 | 100 | 0.56 |
| LMH | 3.54 | 109.1 | 3.3 |
| \mathring{1} | 0.62 | 19.0 | 20.5 |
| \mathring{2} | 0.55 | 17.0 | 34.8 |
| \mathring{3} | 1.50 | 46.2 | 9.0 |
| \mathring{4} | 0.36 | 11.0 | 115 |
| \mathring{5} | 1.99 | 61.2 | 2.3 |
| | 2.31 | 71.0 | 6.5 |
| | 2.80 | 86.2 | 1.7 |
| | 2.08 | 64.0 | 6.4 |

UFH: unfractionated heparin;
LMH: low molecular weight heparin
APTT: activated partial thromboplastin time

The result of Table 1 shows that: all N-desulfated heparin derivatives have significant reduced anticoagulant activity compared with starting heparin. The relative N-sulfate content of No. 3, 5, 6, 7, 8 samples is 46.2%, 61.2% 71.0%, 86.2%, 64.0% and the anticoagulant activity is 1/16, ¼, 1/12, ⅓, 1/11 of heparin respectively. The relative N-sulfate content of No.1, 2, 4 samples is 19.0%, 7.0%, 11.0% and the anticoagulant activity is 1/37, 1/62, 1/205 of starting heparin respectively. It should be noted that the anticoagulant activity of No.4 sample is decreased to 1/205 of starting heparin, this heparin derivative is non-anticoagulant.

Table 2 illustrated the contents of other of Chemical Moieties, including uronic acids, hexosamines, free amino groups, reducing powers and average molecular weights for heparin and the No. 4 sample (N-desulfated heparin) and their corresponding ratios.

TABLE 2
Measurements of chemical moieties of the sample No. 4.

| | Uronic | | TNP- | Reducing | |
|---|---|---|---|---|---|
| Com- | acid | Hexosamine | NH₂ | Power | |
| pound | (%) | (%) | (Abs 348) | (Abs 450) | MW |
| UFH | 26.7 | 23.6 | 0.109 | 0.154 | 19,500 |
| No.4 | 33.0 | 24.9 | 0.838 | 0.179 | 16,100 |
| No.4/UFH | 1.24 | 1.06 | 7.7 | 1.16 | 0.826 |

UFH: unfractionated heparin; No.4: the No.4 N-desulfated heparin sample;
Abs: absorbance in 384 nm;
Mw: average molecular weight (Daltons)

The result of table 2 shows that: The content of uronic acid and hexosamine in heparin chain increases 24% and 6% respectively after the N-desulfated chemical modification. As a result of the N-desulfation, more mounts of free amino acid generates, the No.4 sample contains 7.7-fold free amino acid content compared with heparin. Furthermore, chemical modification makes the chain of the No.4 sample shortened 17.4%, and the average molecular weight decreased from 19500 daltons to 16100 daltons, while the reducing power increased 16%.

In the peritonitis mouse model induced by the intraperitoneal injection of thioglycollate, intravenously administrate LMH and all N-desulfated heparin derivatives to test the inhibition effect of peritoneal infiltration of the inflammation cell including Lymphocytes, monocytes and neutrophils. FIG. 1 shows the result: the anti-inflammation of all the N-desulfated heparin derivatives is better than or equal to the LMH, then this N-desulfated heparin derivatives with 0.1-99.9% N-sulfate of the starting heparin can be used to prevention and treatment of inflammation diseases. Furthermore, intravenously administrate LMH and all N-desulfated heparin derivatives (7.5 mg/kg) to test their effect on the bleeding time. FIG. 2 shows the result, the anticoagulant activity in vivo of No. 1, 2, 4 samples is lower than LMH, while the anticoagulant activity of others is equal to or a little higher than LMH.

Another dose course of bleeding time is carried on to compare the anticoagulant activity of No.4 sample with the LMH intensively, the result is referred to FIG. 3. It demonstrates that intravenously injection of 0.75 mg/kg, 2.5 mg/kg, 7.5 mg/kg, 22.5 mg/kg respectively, the bleeding time of LMH group increases sharply with the adding dose, while the No.4 group has no apparent bleeding time prolonging. Therefore, it can be concluded that No.4 sample has non-anticoagulant activity; the result of the APTT in vivo confirmed this conclusion. As shown in FIG. 4A, intravenous injection of 2.5 mg/kg of heparin, LMWH and the sample No. 4 could trigger 17-fold, 1.6-fold (**, P<0.01 compared with heparin) and 1.1-fold (**, P<0.01 compared with heparin) prolongation of APTT respectively. Following intravenous injections, 7.5 mg/kg LMWH caused 10.7-fold prolongation of APTT while the same amount of the sample No. 4 only induced 2.4-fold (**, P<0.01 compared with LMH) prolongation of APTT (FIG. 4B).

We further compared the anti-inflammatory activities of LMWH and the sample No. 4 (N-desulfated heparin) in the mouse model of acute peritonitis using a lower dosage and a dose course. FIG. 5A shows that intravenous administration of 10 mg/kg LMWH and the sample No. 4 could significantly reduce the peritoneal deposition of neutrophils with the inhibition of 44.9%. The sample No. 4 appeared to be more potent than LMH (15.7% inhibition). This conclusion was further confirmed by the dose courses of LMWH and the sample No. 4 (FIG. 5B). The sample No. 4 was at least 10-fold more potent than LMWH for prevention of neutrophil infiltration in this in vivo model.

We investigated whether the sample No. 4 (N-desulfated heparin) could reduce ischemia and reperfusion injury using a rabbit ear model. FIG. 6A shows that compared to normal ears, complete blockade of blood flow for 6 h followed by spontaneous reperfusion caused significant tissue edema (5.4-fold increase in the ear volume) when they were measured 4 days after the operations. Intravenous injection of 10 mg/kg of the sample No. 4 markedly reduced tissue edema (3.4-fold increase in the ear volume; p<0.05). In contrast, intravenous injections of 3 mg/kg of the sample No. 4 (4.8-fold increase in the ear volumes; p>0.05) and 1 mg/kg of heparin (4.7-fold increase in the ear volumes; p>0.05) had no statistically significant effects on tissue edema. FIG. 6B illustrated the time courses of tissue edema in the operation groups treated with saline and the sample No. 4 (10 mg/kg).

Histological examination and cytologic detection further proved that blocking leukocytes flushing into inflammation sites was one of the anti-inflammation mechanisms for the No.4 sample. Using histological examinations, we found that compared to the tissues from the operation group treated with saline (FIG. 7A), 10 mg/kg the sample No. 4 (FIG. 7B) significantly decreased the deposition of leukocytes within the injured tissues. We also measured the activities of MPO, an enzyme known to be existed exclusively in neutrophils, to represent the amounts of neutrophils in the injured tissues in each group of animals. FIG. 7C shows that compared to the normal tissues, complete blockade of blood flow for 6 h followed by spontaneous reperfusion triggered a substantial amount of neutrophils deposited in the injured tissues. The sample No. 4 (10 mg/kg) significantly inhibited the neutrophil depositions (p<0.05). However, 1 mg/kg heparin (p>0.05) and 3 mg/kg the sample No. 4 (p>0.05) had no statistically significant effects on the neutrophil depositions in the injured tissues after the operations.

FIG. 8 shows that the incidence of tissue necrosis was markedly reduced by treatment with 10 mg/kg the sample No. 4 and less markedly reduced by treatment with 3 mg/kg the sample No. 4. In contrast, the incidence of tissue necrosis was only very moderately reduced after treatment with intravenous administration of heparin (1 mg/kg).

We also investigated the effect of the No.4 sample on acute lung injury using a piglet model. FIG. 9 shows the result of the pathomorphism. Histological examination of normal group indicates homogeneous dilation of alveolus, no apparent edema, hemorrhage, congestion, infiltration of inflammation cell and pathological change coursed by injury of epithelium in small air passage (FIG. 9A). While model group shows extensive edema, petechial hemorrhage, congestion, and collapse of partial lung tissue through macroscopy; through optical microscope we observed apparently congestion of, edema, hemorrhage and infiltration of inflammation cell, heterogeneous dilation, excessive inflation and collapse of alveolus; the desmohemoblast among alveolus broadened (FIG. 9B), as well as mild injury of epithelium in small air passage. In treatment group with the No.4 sample, hemorrhage, congestion, infiltration of inflammation cell was attenuated, exudation in alveolus was decreased, desmohemoblast among alveolus narrowed, homogeneous dilation of alveolus but still mild injury of epithelium in small air passage (FIG. 9C).

A mouse delayed-type hypersensitivity (DTH) model induced by picryl chloride (PCl) was used to detect the effect of the No.4 sample. Table 3 shows the results: The ALT level of the positive control group is markedly increased compared with that of normal group. Treatment with 10 mg/kg and 20 mg/kg No.4 sample markedly deduced the ALT level compared with positive control, the inhibition rate was 93% and 87% respectively, while treatment with 10 mg/ml cyclophosphamide also deduced the ALT level (87% inhibition).

In addition, two model systems in vitro were used to investigate the cellular mechanisms for the anti-inflammatory activity of the No. 4 sample. Our data clearly demonstrate that the No. 4 sample can inhibit adhesion and transendothelial migration of leukocytes, both of which are essential in inflammatory responses. FIG. 10 shows that under a physiological shear stress (2.0 dyne/cm²), adhesion of HL-60 cells to stimulated HUVECs was significantly inhibited by 1 mg/ml the sample No. 4, and the inhibition rate was 78.5% (**, P<0.01). But the same concentration of LMWH only mildly neutralized the adhesion of HL-60 cells under flow (17.4%, *, P<0.05). We then tested whether these compounds could interfere with the transmigration of human neutrophils through the monolayers of HUVECs following TNF-α stimulation. FIG. 11 shows that compared to the unstimulated HUVECs (designated as neutrophils migrated more avidly through the monolayers of TNF-α stimulated HUVECs (designated as +). This increased transmigration of neutrophils could be attenuated by 1 mg/ml of both LMWH and the sample No. 4. The inhibition rate of the No.4 sample was 90.6% (**, P<0.01) while that of LMH was 70% (*, P<0.05). Above data clearly demonstrate that the No. 4 sample can inhibit adhesion and transendothelial migration of leukocytes more potent than LMN, thus the No. 4 sample has a better anti-inflammation effect.

### SUMMARY OF THE INVENTION

In one embodiment, the present invention comprises the preparation of a chemically modified heparin. This chemically modified heparin is defined as an N-desulfated heparin, which results in ˜89% reduction of the N-sulfate content. This compound has significantly reduced anticoagulant activity (more than 99% reduction); it will be referred to as the N-desulfated heparin thereafter.

In a further embodiment, the invention comprises the use of the N-desulfated heparin for prevention and treatment of various acute, subacute and chronic inflammation, conditions characterized by leukocyte infiltration and deposition in the site of tissue injury leading to further damage of the tissues and organs as well as their consequently functional deterioration.

### BRIEF DESCRIPTION OF THE DRAWING

Table 1 illustrates the relative and absolute amounts of N-sulfate and measurements of activated partial thromboplastin time in heparin, Low molecular weight heparin and the chemically modified heparin derivatives.

FIG. 1 shows the effects of intravenous injection of small molecular weight heparin and the chemically modified heparin derivatives on the peritoneal infiltration of total leukocytes, lymphocytes, monocytes and neutrophils in a mouse model of acute peritonitis induced by intraperitoneal injection of thioglycollate.

−: Sterile pyrogen-free saline group

+: Positive control group

LMH represents the low molecular weight heparin therapeutic group 1, 2, 3, 4, 5, 6, 7, 8 represent the N-desulfated heparin groups which contain 19.0%,17.0%,46.2%,11.0%,61.2%,71.0%,86.2%,64.0% N-sulfate respectively.

FIG. 2 shows the effects of the chemically modified heparin derivatives on the bleeding times following intravenous injection of mice.

Saline: Sterile pyrogen-free saline group

LMH: low molecular heparin therapeutic group

1, 2, 3, 4, 5, 6, 7, 8 represent the N-desulfated heparin groups which contain 19.0%,17.0%,46.2%,11.0%,61.2%,71.0%,86.2%,64.0% N-sulfate respectively.

FIG. 3 shows the dose course of the effects of small molecular weight heparin and the N-desulfated heparin (sample No. 4) on the bleeding times following intravenous injection of mice.

FIG. 4 shows the results of aPTT in vivo of heparin, LMWH and sample No. 4 (N-desulfated heparin).

−: Sterile pyrogen-free saline group

Heparin: heparin group

LHM: low molecular group

No.4: the No.4 sample group

FIG. 5 shows the results of the dose course of the No.4 sample and the low molecular heparin in mice peritonitis model (FIG. 5B) and the anti-inflammation activity at the dose of 10 mg/kg (FIG. 5A).

−: Sterile pyrogen-free saline group

+: Positive control group

LHM: low molecular group

No.4: the No.4 sample group

FIG. 6 shows the effect of the No.4 sample in rabbit ear ischemia and reperfusion injury model.

FIG. 6A shows the effect on the day 4 after the operation:

−: Normal group

+: Positive control group

Heparin: heparin treatment group at dose of 1 mg/kg

No.4(3): the No.4 sample group at dose of 3 mg/kg

No.4(10): the No.4 sample group at dose of 10 mg/kg

FIG. 6B shows the time curve of the effect of the No.4 sample:

Saline: Sterile pyrogen-free saline group

No.4: the No.4 sample group

FIG. 7 shows the histological examinations of the effect of the No.4 sample in rabbit ear ischemia and reperfusion injury model.

FIG. 7A shows the result of leukocytes staining within the injured tissues:

Saline: Sterile pyrogen-free saline group

No.4: the No.4 sample group

FIG. 7B shows the result of detecting the amounts of neutrophils in the injured tissues:

−: Normal control group

+: Positive control group

Heparin: heparin treatment group at dose of 1 mg/kg

No.4(3): the No.4 sample group at dose of 3 mg/kg

No.4(10): the No.4 sample group at dose of 10 mg/kg

FIG. 8 shows the result of that the sample No. 4 markedly reduced the incidence of tissue necrosis.

+: Positive control group

Heparin: heparin treatment group at dose of 1 mg/kg

No.4(3): the No.4 sample group at dose of 3 mg/kg

No.4(10): the No.4 sample group at dose of 10 mg/kg

FIG. 9 shows the effect of the No.4 sample in the

A: Normal control group

B: Model control group

C: treatment group of the No.4 sample

FIG. 10 shows that the No.4 sample could inhibit adhesion of leukocytes to endothelial cells:

−: Sterile pyrogen-free saline group

+: Positive control group

EDTA: a chelator for divalent cations

LHM: low molecular group

No.4: the No.4 sample group

FIG. 11 shows the result of the No. 4 sample inhibiting the transendothelial migration of leukocytes.

−: Sterile pyrogen-free saline group

+: Positive control group

LHM: low molecular group

No.4: the No.4 sample group

### DETAILED DESCRIPTION OF THE INVENTION

In accordance with the present invention, a method for treatment of inflammation is provided, which comprises administration of a therapeutically effective amount of the N-desulfated heparin. In the method of the present invention, the term “therapeutically effective amount” means the total amount of the N-desulfated heparin that is sufficient to show a meaningful patient benefit, that is, healing of pathological conditions characterized by leukocyte infiltration and deposition or increase in rate of healing of such conditions, whether administered in combination, serially or simultaneously.

In practicing the method of treatment of this invention, a therapeutically effective amount of the N-desulfated heparin is administered to a mammal having a disease state. Such disease states include inflammatory disorders such as various kinds of arthritis, asthma, dermatitis and psoriasis, acute respiratory distress syndrome, ulcerative colitis, various types of hepatitis, ischemia/reperfusion injury (including myocardial, renal, skeletal muscular, intestinal, cerebral and pulmonary ischemia/reperfusion injury), shock, severe trauma and transplant rejection.

In practicing the method of the present invention, the N-desulfated heparin may be administrated alone or in combination with other therapies. For example, the non-anticoagulant heparin may optionally be used in combination with certain cytokines, lymphokines, or other hematopoietic factors such as M-CSF, GM-CSF, NKSF, IL-1, IL-2, IL-3, IL-4, IL-5, IL-6, IL-7, IL-8, IL-9, IL-10, IL-11, IL-12, G-CSF, Meg-CSF, and erythropoitin to treat inflammatory states. It is contemplated that the method of treatment will allow the non-anticoagulant heparin to synergize with the cytokine, lymphokine, or other hematopoietic factor, thereby augmenting the anti-inflammatory response. Alternatively, the method of treatment will allow the N-desulfated heparin to minimize the potential side effects caused by the cytokine, lymphokine, or other hematopoietic factor.

Pharmaceutical compositions used to practice the method of the present invention may contain, in addition to the N-desulfated heparin, pharmaceutically acceptable carries, diluents, fillers, salts, buffers, stabilizers, and/or other materials well known in the art. The term “pharmaceutically acceptable” means a non-toxic material that does not interfere with the effectiveness of the biological activity of the active ingredient(s). The characteristics of the carrier or other material will depend on the route of administration.

Administration of the N-desulfated heparin used to practice the method of the present invention can be carried out in a variety of conventional ways, such as oral ingestion, or cutaneous, subcutaneous, or intravenous injection. Intravenous administration to the patient is preferred.

When a therapeutically effective amount of the N-desulfated heparin is administered orally, the non-anticoagulant heparin will be in the form of a tablet, capsule, powder, solution or elixir. When administrated in tablet form, the pharmaceutical composition of the invention may additionally contain a solid carrier such as a gelatin or an adjuvant. The tablet, capsule, and powder contain from about 5 to 95 % the N-desulfated heparin, and preferably from about 25 to 90% the N-desulfated heparin. When administered in liquid form, a liquid carrier such as water, petroleum, oils and animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the N-desulfated heparin, and preferably from about 1 to 50% the N-desulfated heparin.

When a therapeutically effective amount of the N-desulfated heparin is administered by intravenous, cutaneous or subcutaneous injection, the N-desulfated heparin will be in the form of a pyrogen-free, parentally acceptable protein solutions, having due regard to pH, isotonicity, stability, and the like, is within the skill in the art. A preferred pharmaceutical composition for intravenous, cutaneous, or subcutaneous injection should contain, in addition to the N-desulfated heparin, an isotonic vehicle such as Sodium Chloride Injection, Ringer [symbol] Injection, Dextrose Injection, Dextrose and Sodium Chloride Injection, Lactated Ringer [symbol] Injection, other vehicle as known in the art. The pharmaceutical composition used to practice the method of the present invention may also contain stabilizers, preservatives, buffers, antioxidants, or other additive known to those of skill in the art.

The amount of the N-desulfated heparin in the pharmaceutical composition used to practice the method of the present invention will depend upon the nature and severity of the condition being treated, and on the nature of prior treatments which the patient has undergone. Ultimately, the attending physician will decide the amount of the N-desulfated heparin with which to treat each individual patient. It is contemplated that the various pharmaceutical composition should contain about 0.1 μg to about 100 mg of the N-desulfated heparin per kg body weight.

The duration of intravenous therapy using the pharmaceutical composition used to practice the method of the present invention will vary, depending on the severity of the disease being treated and the condition and potential idiosyncratic response of each individual patient. It is contemplated that the duration of each application of the N-desulfated heparin will be in the range of 12 to 24 hours of continuous intravenous administration. Ultimately the attending physician will decide on the appropriate duration of intravenous therapy using the pharmaceutical composition of the present invention.

The pharmacokinetics and the toxicity experiments of the N-desulfated heparin are being carried now.

### EXAMPLE 1

### Chemical Modification of Heparin

Preparation of Pyridine Salt of Heparin

The sodium salt of heparin (Sigma) was passed through a column of Dowex (Strongly Acidic Cation Exchanger 50×4-100; Sigma) and the effluent was neutralized with pyridine. It was then lyophilized to give a white powder [Inoue and Nagasawa, Carbohydr. Res. 46, 87-95 (1976)].

Preparation of N- and O-Desulfated Heparins

The pyridine salt of heparin (100 mg dissolved in 0.25 ml of double destined and deionized H₂O) was mixed with 4.75 ml of dimethylsulfoxide (DMSO; Sigma) at 50° C. for 2 hour (sample No. 4), 1 hour (sample No. 1), or 20° C. for 3 hours (sample No. 3). The sample was diluted with an equal volume of H₂O and the reaction was terminated by adjusting pH to 7.0 with 0.1 NNaOH [Nagasawa and Inoue, Carbohydr. Res. 36, 265-271 (1976); Inoue and Nagasawa, Carbohydr. Res. 46, 87-95 (1976); Tiozzo et al., Thromb. Res. 70, 99-106 (1993)].

The sample No.1 heparin was further dissolved in 1 NNaOH (4% heparin concentration) at 60° C. for 4 hours followed by neutralization to pH 7.0 with 1 N HCl (sample No. 2). The pyridine salt of heparin was dissolved in 1 NNaOH (4% heparin concentration) followed by treatment at 40° C. (sample No. 5) or at 60° C. (sample No. 6) for 4 hours. Further, the sodium salt of heparin was dissolved in 1 N NaOH (4% heparin concentration) followed by treatment at 40° C. (sample No. 7) or at 60° C. (sample No. 8) for 4 hours. Sample was then neutralized to pH 7.0 with 1 N HCl [Tiozzo et al., Thromb. Res. 70, 99-106 (1993); Lloyd et al., Biochem. Pharmacol. 20, 637-648 (1971)].

Preparation of Final Heparin Derivatives

After chemical modification, these heparin derivatives were individually passed through a column of Amberlite IRA-400 (Strongly Basic Anion Exchanger; Sigma) to remove the free sulfate ions. The effluent was neutralized to pH 7.0 with 1 N NaOH. It was then desalted by passing through a column of Bio-Gel P-2 (Bio-Rad). Heparins were monitored at the optical densities of 214 nm and 230 nm.

### EXAMPLE 2

### Measurements of Activated Partial Thromboplastin Time

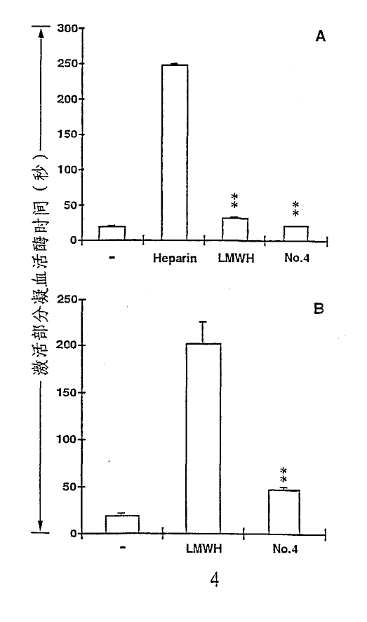

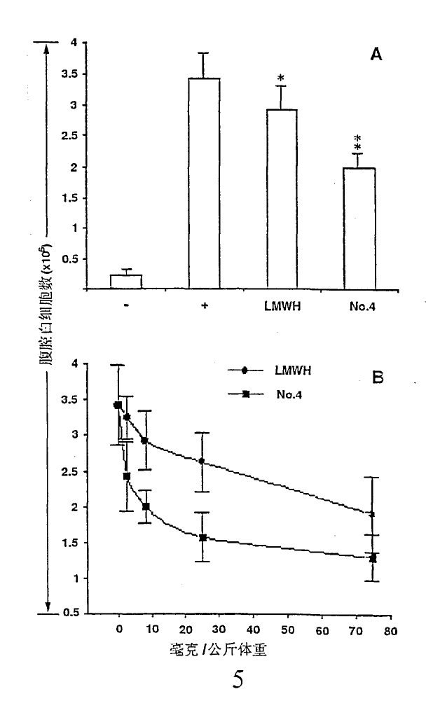

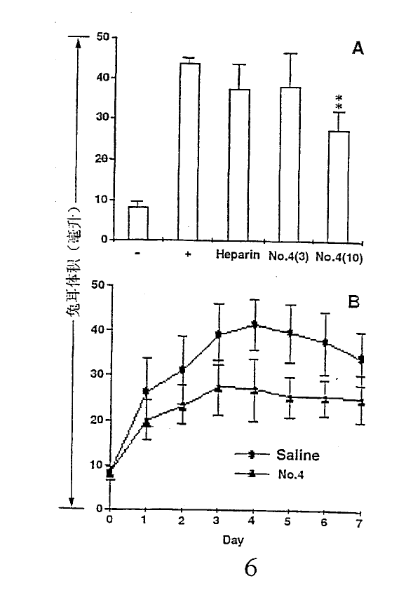

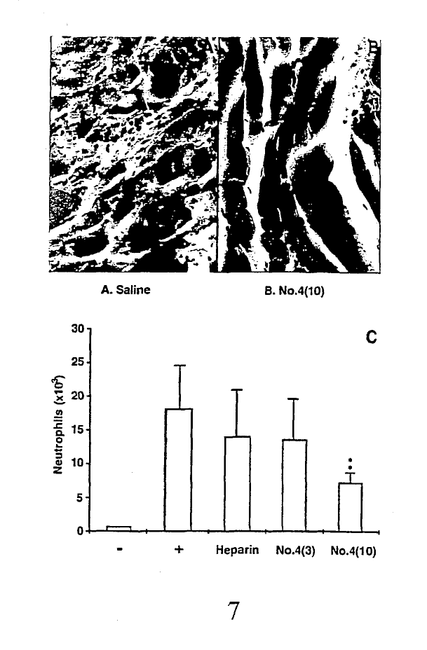

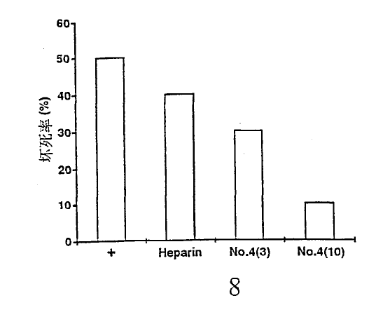

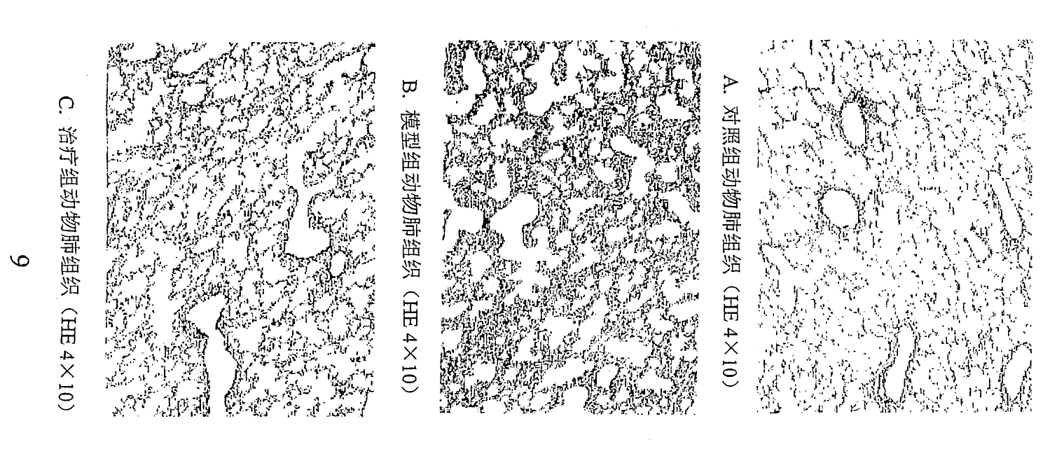

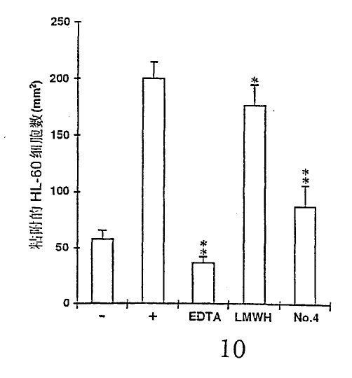

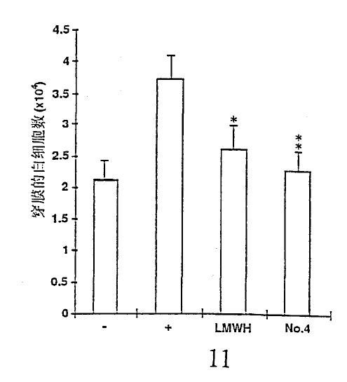

Activated partial thromboplastin time (APTT) was measured using fresh human blood from healthy volunteers. The known amounts of heparin, Low molecular weight heparin and the chemically modified heparin derivatives were added prior to the determination of APTT using Silimat™ (bioMeieux sa) as activator. Six assays were performed for each compound and the anticoagulant activity was expressed as the concentration (ug/ml) that doubles the aPTT time (2-aPTT). The higher concentration is parallel to the lower anticoagulant activity (Table 1). The same assay was carried to determine the aPTT time of the mice in vivo (FIG. 4.).

### EXAMPLE 3

### Measurement of N-Sulfate Amounts

Solution and reagents: 5% sodium nitrite, 33% acetic acid, 3.8% trichloroacetic acid, barium chloride-gelatin reagent (prepared by dissolving 1 g of gelatin in 100 ml of water, incubated at 60° C. to make a complete dissolution, then put it at 4° C. overnight. The mixture was filtered after adding 0.5 g of barium chloride and was ready to use after standing for 4 hours at room temperature. This reagent was stored at 4° C. and could be used for about one week.)

N-sulfates in heparin and various chemically modified heparin derivatives were determined by nitrous acid treatment as described [Inoue and Nagasawa, Anal. Biochem. 71, 46-52 (1976)]. Briefly, 0.5 ml of a sample solution was mixed with 0.5 ml of 5% sodium nitrite and 0.5 ml of 33% acetic acid. After shaking, the mixture was incubated at room temperature for 30 min and 4.5 ml of 3.8% trichloroacetic acid was then added. After shaking again, 1.5 ml of the barium chloride-gelatin reagent was added. Following shaking again immediately, the sample was left standing for 20 min and the turbidity of the sample was measured at 500 nm. The absolute N-sulfate mounts of all N-desulfated heparin sample were calculated with K₂SO₄ as control, while the relative N-sulfate mounts of all N-desulfated heparin were calculated using the starting heparin as 100%.

The contents of uronic acid of the No.4 sample and heparin were determined according to the method of Bitter and Muir (Anal. Biochem. 4:330-334,1962). The hexosamine contents were determined according to the method of Elson and Morgan (Biochem. J. 27:1824-1828, 1993). The contents of free amino group, at a 2 mg/ml concentration, were determined as before (Yoshizawa et al., Biochim. Biophys. Acta., 141:358-365,1967). Reducing power, at a 2 mg/ml concentration, was measured using the 3, 6-dinitrophthalic acid method (Momose et al., Talanta, 4:33-37,1960). The average molecular weights were determined using the end group analysis (Hopwood and Robinson, Biochem. J. 135:631-637,1973).

### EXAMPLE 4

### Anti-Inflammatory Heparin Screening Assay in vivo

Balb/c mice (males, 5 weeks old, 20±1 g body weight) were purchased from Shanghai Animal Center of Chinese Academy of Sciences. Negative control group contained 8 mice, they were intraperitoneally injected with 1 ml of sterile pyrogen-free saline. Fifteen minutes later, mice were intravenously injected with 0.2 ml sterile pyrogen-free saline alone. The positive control group (12 mice) was intraperitoneally injected with 1 ml of 3% thioglycollate broth. Fifteen minutes later, mice were intravenously injected with 0.2 ml saline. The mice of the anti-inflammation group (7-11) were intraperitoneally injected with 1 ml of 3% thioglycollate broth. Fifteen minutes later, mice were intravenously injected with saline containing 1.5 mg of low molecular weight heparin or any of the chemically modified heparin derivatives. Mice were sacrificed at 2 hours. The peritoneal cavities were lavaged with 8 ml of ice-cold PBS containing 10 U/ml of heparin to prevent clotting. Centrifuged at 1500 rpm for 5 mins. The total peritoneal leukocytes and their differentiation (lymphocyte, monocyte and granulocyte) were measured using Cell-Dyn1700 (Abbot Laboratories, USA).

### SUMMARY

The mice of heparin therapeutic groups appear more active than the positive control group during the experiments time.

Shortly after the injection of the samples, few mounts of blood was bleeding from the pinpricks of the mice administrating the No.4 sample, concomitantly the coagulant time was shorter than those groups administrating other heparin samples. Furthermore, when collecting the total peritoneal infiltrated leukocytes, the group administrating No.4 sample showed no inner bleeding phenomena, but other groups has little or more bleeding. The experiments watching above reflected the No.4 sample has significant reduced anticoagulant activity while remaining the anti-inflammation effect.

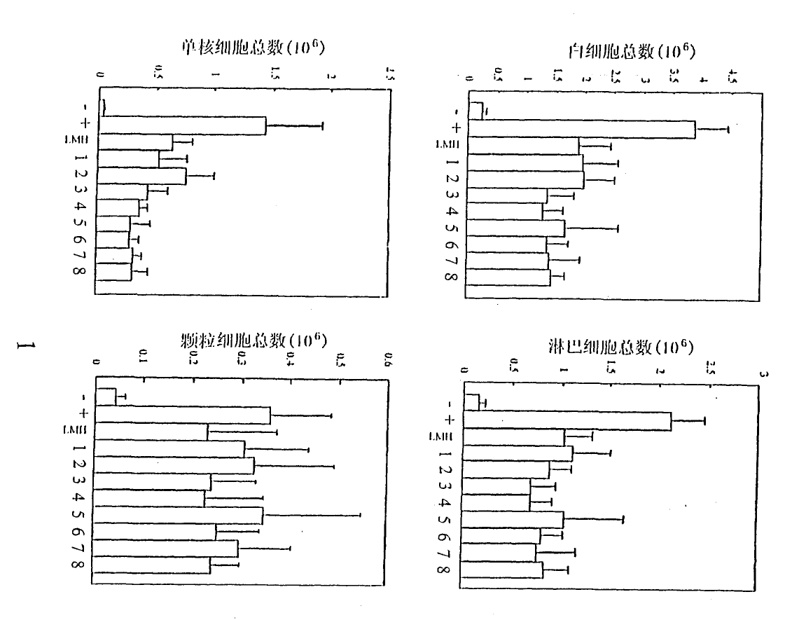

FIG. 1 shows the result of the anti-inflammation screening assay in vivo, at the same time table 2 represents the inhibition percent of the peritoneal infiltrated inflammation leukocytes.

TABLE 2
The inhibition percent of inflammation cell infiltration in
the mice peritonitis model.

| | LMH(%) | 1(%) | 2(%) | 3(%) | 4(%) | 5(%) | 6(%) | 7(%) | 8(%) |
|---|---|---|---|---|---|---|---|---|---|
| WBC | 54.4 | 52.8 | 52.1 | 68.8 | 70.9 | 60.6 | 69.1 | 68.0 | 66.9 |
| Lymphocyte | 54.9 | 49.5 | 62.0 | 72.1 | 72.1 | 59 | 65.9 | 68.0 | 64 |
| Monocyte | 56.2 | 64.7 | 47.6 | 71.2 | 76.3 | 81.7 | 82 | 79.9 | 80.3 |
| Granulocyte | 40.3 | 16.1 | 11.2 | 36.9 | 40.9 | 4.6 | 33.4 | 19.3 | 10.6 |

LMH: Low molecular heparin;
1,2,3,4,5,6,7,8 is the same heparin derivatives samples to table 1.

The inhibition percent is got through this calculating formula:

$$1 - \frac{\begin{matrix}( Cell number of all heparin derivatives - \\ Cell number of negative control )\end{matrix}}{\begin{matrix}( Cell number of positive control - \\ Cell number of negative control )\end{matrix}}$$

All N-desulfated heparin has different anti-inflammation activity, the result showed that the total white blood cell inhibition percent is about 52.1-70.9%, total Lymphocyte inhibition percent is about 49.5-72.1%, monocyte inhibition percent is about 47.6-82%, granucyte inhibition is about 10.6-40.9%, all N-desulfated heparin has better anti-inflammation activity than LMH which is commonly used in the art, besides, N0.4 sample is the best.

### EXAMPLE 5

### Anticoagulant Activity Screening Assay in vivo

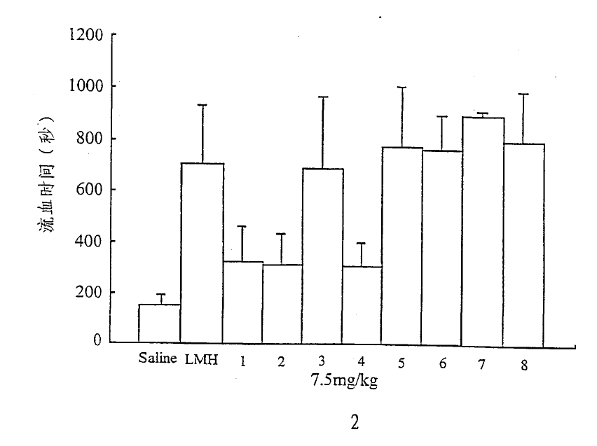

Swiss mice (five weeks old, weigh 16 g) were purchased from Shanghai Animal Center of Chinese Academy of Sciences. Each group contained 9-11 mice. All the experiments were carried on under 25±1° C., because the bleeding time was varied according to environment temperature. Bleeding times of mice were measured exactly as described previously (Dejana et al., 1982). Low molecular weight heparin and the chemically modified heparin derivatives (all at 0.12 mg/mouse; 7.5 μg/gram of body weight) were injected into each Swiss mouse 15 min prior to the tail cutting (2 mm from the tail tip) with a razor blade. For determination of bleeding time, the amputated tail was sunk longitudinally in phosphate buffered saline, pH 7.4, at 25° C. Complete clotting was recorded after stopping the bleeding for 30 sec. And if the bleeding time was longer than 15 min, it was counted as 15 min. FIG. 2 shows the result: at the condition of injection of 120 ug sample per mouse, No.1, No.2, No.4 samples have significant shorter bleeding time than LMH, little longer that the saline group.

### EXAMPLE 6

### Dose Course Assay of the Anticoagulant Activity of the No.4 Sample

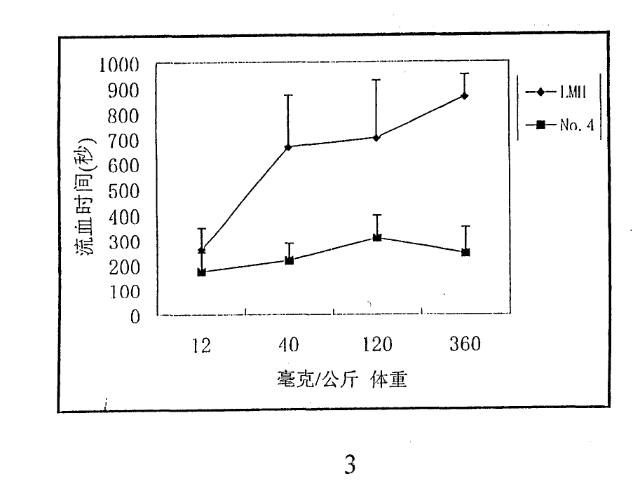

The assay and the experiment condition were according to that of example 5. The No.4 sample and LMH were injected into the Swiss mice with the mounts of 0.75 mg/kg, 2.5 mg/kg, 7.5 mg/kg and 22.5 mg/kg respectively to test the bleeding time. FIG. 3 shows the result, with the administration mounts of LMH increasing, the bleeding time prolongs sharply, while the No.4 group has no apparent prolonging. Therefore, we can say: No.4 sample has non-anticoagulant activity.

### EXAMPLE 7

### Effect of the No.4 Sample in the Rabbit Ischemia and Reperfusion Injury Model

Ischemia and reperfusion injury assay. New Zealand albino rabbits were purchased from Shanghai Animal Center of Chinese Academy of Sciences. Each group contains 6 rabbits. General anesthesia was induced and maintained by peritoneal injection of pentobarbital sodium (45 mg/kg). After subcutaneous injections of lidocaine at the bases of rabbit ears to block the supplemental local nerves, both ears were carefully amputated at their bases in sterile conditions under a surgical microscope. Only the central artery, the central vein, and a small non-vascular cartilage bridge were left intact. The ear's sensory nerves were transacted to render the ears in a permanently anesthetic condition and the ears were approximated to their bases with suture. A non-traumatic microvascular clip was then placed on the central artery of each ear to stop the blood flow for complete ischemia. After 6 h, the clip was removed and the ear was allowed to spontaneous reperfusion (Mihelcic et al., 1994;Lee et al., 1995; Han et al., 1995). For the therapeutic intervention, one bolus of intravenous administration of saline alone, saline with heparin or the No. 4 sample was given at the beginning time of reperfusion (removal of the microvascular clip).

Measurements of tissue edema and necrosis. Ear volume (as a reflection of tissue edema) was measured daily for seven continuous days following removal of the microvascular clip. Ear volume was quantified by determination of the volume displacement after inserting the amputated portion of the ear (to the level of the suture line) into a fluid-filled vessel. Tissue necrosis was assessed, in a double-blind manner, by the presence or absence of tissue necrosis (defined as >5% skin sloughing of the total ear surface) on day 7 (Han et al., 1995).

Mycloperoxidase (MPO) assay. MPO activities were measured as previously described (Geng et al., 1990; Schierwagen et al., 1990; Mihelcic et al., 1994). The ear tissues (no skins) were surgically taken 24 h after reperfusion. They were weighed and placed (0.5 g/ml) in 50 mM potassium phosphate buffer, pH 6.0, supplemented with 0.5% hexadecyltrimethyl-ammonium bromide (Sigma). They were subsequently homogenized by freeze-thaw three times and sonication twice. The mixtures were then centrifuged at 10,000 g for 10 min. The supernatants were heated at 60° C. for 2 h to inactivate potential inhibitors of MPO. For generation of standard curves, fresh blood was taken from healthy rabbits and rabbit neutrophils were isolated using Ficoll-Paque Plus (Amersham Pharmacia Biotech) according to the manufacturer's instructions. These isolated neutrophils were more than 95% pure based on differential staining of leukocytes.

Tissue histology. Tissues were taken by the 3-mm punch biopsy from the anesthetic ears 24 h after the operation. Samples were fixed by immersion in Bouin's fixation solution (75% picric acid, 24% formaldehyde, 1% acetic acid) for 24 h at 24° C. followed by paraffin embedding. Tissue sections (5-μm thick) were subsequently dewaxed and stained with hematoxylin and eosin. They were photographed at ×280.

### EXAMPLE 8

### The Effects of No.4 Sample on Acute Lung Injury

17 male piglets (6.0-7.4 kg body weight, average 6.7 kg) were purchased from Shanghai Pasturage Institute, Shanghai Academy of Agricultural Sciences (SAAS). The piglets were abrosiaed for 12 hours. Before the operation the piglets were intramuscularly injected with prazosin (0.02 mg/kg) and sedated with Ketamine Hydrochloride (7 mg/kg, made by Shanghai Middle West Pharmaceutical Co. Ltd). Fifteen minutes later the piglets and the scarf skin for operation and intubation were cleaned. After the vein bypass established the piglets were given intravenous injection of with 2.5 mg/kg propofol (Fresenius, Germany) to induce anesthesia. The piglets were put on the infrared constant temperature table (YQT-2, made by Shanghai) on their back. Then 0.15 mg/kg Vecuronium Bromide (made by China Xianju Pharmaceutical company, Ltd.) was given (i.v.) to relax the muscle. A ballonet catheter was inserted through the mouth and it was linked to a respiration machine (Siemens, 900C). After conventional sterilization and laying sheets the right arteriae femoralis was separated. And 24G cannulas were left for artery blood sample collection. The femoral were linked to hemodynamics equipments, which could monitor the artery blood pressure and rhythm of heart. Thirty minutes later all items indicating the basic condition were detected. The piglets were divided into three groups and were operated according to the method. After the operation anesthesia were stopped and the piglets came round naturally. The trachea cannulas were withdrawn when they could breath independently, PaO₂ (partial pressure of oxygen) was lower than 40 mmHg, SaO₂ (oxygen saturation) was higher than 95 percent and could react to pain stimulation. Then the piglets were put in incubator where the temperature was maintained 21 centigrade. The piglets were intramuscularly injected with Bucinnazine Hydrochloride to ease pain and were given (i.v.) glucose-lactate cycle solution (10%, 5 ml/kg/h). Twenty-four hours later the piglets were induced anesthesia by intravenous injection and intubated into trachea again. The speed of injection was adjusted to 10-15 ml/kg/h according to heart rate, CVP (central venous pressure) and SAP (systemic arterial pressure). After sterilization an incision was made in left femoral and a 24G cannula was inoculated for blood sample collection and hemodynamics monitoring. An incision was made in left venae jugulalis extema and an 18G catheter was left for CVP monitoring and venous blood sample collection. When the condition of the piglets was stable the right-down part on the abdomen was sterilized and laid sheet again. After different operations depending on different group the piglet belong the incision was closed. The piglets were given intravenous injection of gentamicin (20,000 units) to prevent inflammation. When ALI appeared the piglets were treated according to requirements of each group. Sputum was suctioned every 2 hours during the experiment. 1 mg/kg propofol and 0.05 mg/kg Vecuronium Bromide were once injected discontinuously to maintain the breath frequency at 40-50 times per minute and V_{E} at 0.3L/min/kg. The piglets were treated with 5% NaHCO₃ solution in case acidosis emerged. When SAP was lower than 60 mmHg, the piglets were given (i.v.) dopamine to maintain the blood pressure. At the end of the experiment the piglets were sacrificed with 10 ml 10% KCl solution (i.v.).

The piglets were divided into three groups at random before the experiment.

Group A (Control Group, n=5): The piglets were made an incision at the right-lower abdomen (3 cm) and the intestine were agitated before the incision was closed. Twenty-four hours later the incision was opened again and the intestine was agitated again. The incision was closed after the peritoneal cavity was washed with 200 ml saline (37C). The animals were observed for 12 hours.

Group B (Model Group, n=6): An incision at the right-lower abdomen (3 cm) was made followed by a 2-cm perforation in the distal cecum (5 cm from the end). It was sewed and 0.5 cm mucosa was evaginated in order to form an ostium with a diameter of 2 cm. The incision was then closured surgically. Twenty-four hours later, the incision was opened again and the ostium was sewn up. The incision was eventually closured after washing the peritoneal cavity with 200 ml saline.(37° C.). The piglets were treated with machinery gassing after ALI emergence.

Group C (Therapy Group, n=6): All procedures were exactly identical to Group B except that 12 mg/kg No.4 sample was intravenously administered in early phase of acute lung injury.

Criterion for Determination of ALI:

A. PaO₂/FiO₂<300 mmHg (PaO₂: Partial pressure of oxygen; FiO₂: Fraction inspired oxygen concentration)

B. Pulmonary dynamics compliance (Cdyn) is lower than that in basic condition by 30%.

Pathology Observation:

Perfusion fixation of lung: Piglets were heparinized, the thorax was opened and the left and right main bronchia were separated. The left lung was inflated with air pressure of 30 cmH₂O. One minute later some air was let out to keep the pressure at about 10 cmH₂O. Meanwhile the right ventricle and the left atria were opened and perfused with 4% formalin at pressure of 65 cmH₂O for 30 minutes.

The left lung was kept in 4% formalin. Three days later lung tissue was taken at 0, 2, 6 and 9 point respectively in order to minimize the effect of gravity on the pathological change. Tissue samples were subsequently dehydrated, embedded with paraffin, sliced up and stained with hematoxylin and eosin (HE).

Tissue sections were observed with optical microscope and were classified into 5 grades according to edema, bleeding, infiltration of inflammation cell, pathological change coursed by epithelium injury of small air passage: 0 grade means normal; 1 grade means that the pathological change is light and area limited; 2 grade means that the pathological change is middling and limited; 3 grade that the pathological change is middling but extensive or remarkable at some part; 4 grade means that the pathological change is remarkable and extensive.

### EXAMPLE 9

### The Effects of No.4 Sample on the Mouse Model of Acute Liver Injury

Hypersensitivity on Swiss mice (18-22 g body weight) were induced by smearing 1% (w/v) PCl in absolute ethanol solution on abdomen after the ventral hair razed for 5 consecutive days. After another five days, 0.5% PCl olive oil solution (Sigma Chemical Co. St. Louis, Mo.) was intrahepatically injected. Blood samples were collected 18 hours for the measurements of alanine aminotransferase (ALT). Mice were intraperitoneal administered twice either saline, No. 4 sample (10 or 20 mg/kg) or cyclophospamide (10 mg/kg) at 0 and 5 hours after PCl intrahepatically injection. Table 3 shows the results: The ALT level of the positive control group is markedly increased compared with that of normal group. Treatment with No.4 (both dosages) or cyclophosphamide markedly deduced the ALT level compared with positive control.

TABLE 3
The effects of No. 4 sample on acute liver injury in the PCl-DTH mice

| | Dosage | Number | ALT |
|---|---|---|---|
| Group | (mg/kg) | of mice | (Karmen unit) |
| Normal | — | 5 | 21.9 ± 12.7 |
| Positive control | — | 8 | 172.6 ± 78.2** |
| No.4 sample | 10 | 10 | 32 ± 15.9^{##} |
| No.4 sample | 20 | 9 | 40.3 ± 36.2^{##} |
| Cyclophosphamide | 10 | 8 | 40.4 ± 9.4^{##} |

**P < 0.01 (compared with normal),
^{##}P < 0.01 (compared with positive control).

### EXAMPLE 10

### Laminar Flow Assay of the No.4 Sample Inhibiting Adhesion of Leukocytes to Endothelial Cells

Human umbilical vein endothelial cells (HUVECs; less than three passages) were cultured on one-well chamber slide (Nalge Nunc, Naperville, Ill.) pre-coated with 1% gelatin as previously described (Geng et al., 1990; Asa et al., 1995). For cytokine stimulation, monolayers of HUVECs were incubated with 300 units/ml of tumor necrosis factor-α (TNF-α; Promega, Madison, Wis.) for 12 h. Slides were mounted in a flow chamber as before (Ma and Geng, 2000).

Human promyeloid HL-60 cells (CCL 240) were purchased from American Tissue Culture Collection (Rockville, Md.). They were cultured in RPMI 1640 medium supplemented with 10% heat inactivated newborn bovine calf serum (BCS), 4 mmol/L L-glutamine, 100 units/ml penicillin and 100 μg/ml streptomycin at 37° C. in the presence of 5% CO₂.

After washing once with PBS, HL-60 cells were resuspended at 0.5×10⁶/ml in PBS supplemented with 10 mmol/L HEPES, pH 7.4, and 2 mmol/L CaCl₂ in the presence of heparin, LMWH and the sample No. 4 at 22° C. for 15 min. They were then precisely injected through the flow chamber at 22° C. using a syringe pump. The wall shear stress used was 2.0 dyne/cm². The numbers of bound cells were quantified from videotape recordings of 10-20 fields of view obtained (3-4 min after flowing cells through the chamber).

### EXAMPLE 11

### Effect of the No.4 Sample on Preventing the Transmigration of Human Neutrophils Through the Monolayers of the Stimulated HUVECs

HUVECs (5×10⁴ cells/well) were seeded in the upper chambers of 24-well Transwell® plates (Costar, Cambridge, Mass.) pre-coated with 1% gelatin. For cytokine stimulation, monolayers of HUVECs were incubated with 300 units/ml of TNF-α for 12 h (Geng et al., 1997). Fresh human blood was obtained from healthy volunteers according to the regulations of Chinese Academy of Sciences. Human neutrophils were isolated using Ficoll-Paque Plus (Amersham Pharmacia Biotech, Shanghai, China) according to the manufacturer's instructions. The isolated neutrophils were more than 95% pure based on differential staining of leukocytes. After washing the upper chambers twice, human neutrophils (2.5×10⁶ cells/ml; more than 95% purity) were resuspended in serum-free M199 medium, in the presence of heparin, LMWH and the sample No. 4, and added to the upper chambers of the Transwell plates for 1 h. The upper wells were then removed and the cells sticking on the lower surface of the filters were released by repeated pipetting. Cells in the lower chambers were counted using a hemocytometer.

1. A method of using the N-desulfated heparins for prevention and treatment of acute, subacute and chronic inflammation, said the relative N-sulfate content of the N-desulfated heparins is 0.1-99.9%.

2. The method of claim 1 wherein said the N-desulfated heparin can be administered alone.

3. The method of claim 1 wherein said the N-desulfated heparin can be used in combination with certain cytokines, lymphokines, or other hematopoietic factors such as M-CSF, GM-CSF, NKSF, IL-1, IL-2, IL-3, IL-4, IL-5, IL-6, IL-7, IL-8, IL-9, IL-10, IL-11, IL-12, G-CSF, Meg-CSF, and erythropoitin to treat inflammatory states.

4. The method of claim 1 wherein said the pharmaceutical compositions used to practice the method of the present invention may contain, in addition to the N-desulfated heparin, pharmaceutically acceptable carries, diluents, fillers, salts, buffers, stabilizers, and/or other materials.

5. The method of claim 1 wherein said the N-desulfated heparin can be administered by oral ingestion, or cutaneous, subcutaneous, or intravenous injection. Intravenous administration to the patient is preferred.

6. The method of claim 1 wherein said the intravenous administration of N-desulfated heparin to the patient is preferred.

7. The method of claim 6 wherein said the duration of each application of the N-desulfated heparin will be in the range of 12 to 24 hours of continuous intravenous administration.

8. The method of claim 5 wherein said N-desulfated heparin, when orally administered, can be in the form of a tablet, capsule, powder, solution or elixir.

9. The method of claim 8 wherein said when orally administrated the tablet, capsule, and powder contains from about 5 to 95% the N-desulfated heparin.

10. The method of claim 8 wherein said when administered in liquid form, the pharmaceutical composition contains from about 0.5 to 90% by weight of the N-desulfated heparin.

11. The method of claim 1 wherein said the pharmaceutical composition should contain about 0.1 μg to about 100 mg of the N-desulfated heparin per kg body weight.
